(19) United States
(12) Patent Application Publication
(10) Pub. No.: US 2006/0153776 A1
(43) Pub. Date: Jul. 13, 2006

(54) Echogen microparticles essentially used as a contrast agent for ultrasound exploration and/or as emboli for ultrasound detection

(76) Inventors: Celine Chaix, Levallois-Perret (FR); Nathalie Lassau, Saint-Mande (FR); Angelo Paci, Paris (FR); Pierre Peronneau, Chateauneuf En Thymerais (FR); Philippe Reb, Chaumes En Brie (FR); Alain Roche, Buc (FR); Valerie Rouffiac, Rosny-Sous-Bois (FR)

Correspondence Address: IP GROUP OF DLA PIPER RUDNICK GRAY CARY US LLP, 1650 MARKET ST, SUITE 4900, PHILADELPHIA, PA, 19103, US

(21) Appl. No.: 10/543,660

(22) PCT Filed: Jan. 28, 2004
(86) PCT No.: PCT/FR04/00193

§ 371 Date: Oct. 4, 2005

(30) Foreign Application Priority Data
Jan. 29, 2003 (FR) 03/01008

(51) Int. Cl. A61K 49/22

(52) U.S. Cl. 424/9.51

(57) ABSTRACT

A composition for ultrasonic exploration including echogenic microparticles including a macromolecular network of reticulated hydrophilic polymers and/or copolymers which micro-particles have a diameter between 0.1 μm and 10 μm and are obtained from an inverse mini-emulsion of identical or different monomers or polymers.

The present invention relates to microparticles and their methods of manufacture. The invention is concerned in particular but not exclusively with the use of these microparticles as contrast agent for ultrasonic exploration.

The physical principle at the base of an echographic or Doppler examination is the backscattering of ultrasounds by the tissues. The examination probe generates a pulsed mechanical wave with a given energy (calculated in terms of mechanical index) at a frequency classically comprised between 5 to 13 MHz (megahertz). This wave is then propagated down from the surface of the probe into the tissues. The laws of reflection are applied at each ultrasonic interface encountered and a part of the energy is returned toward the probe whereas the remaining energy continues its downward path. The term “ultrasonic interface” denotes here any rupture of acoustic impedance between two environments. This impedance is defined as the product of the propagation speed of the ultrasounds by the volumetric mass of the environment under consideration. In other words, when the wave arrives at the interface between two different structures, e.g., between a muscle and a bone, a “backscattered” wave is returned. The energy of this wave is a function of the difference of impedance between these two environments. The more significant this differential, the more the returned energy will be elevated. In terms of image reconstruction, this backscattered energy will be coded by a level of gray. Thus, two pieces of information are available from one echographic image:

1) The localization in depth of tissue structures by means of the pulsatility of the wave;

2) An evaluation of the tissue density by the level of gray represented.

It is possible to superpose a color image on the gray-level image. This new representation permits the localization of the structures that are no longer fixed but mobile such as erythrocytes flowing in the vessels. The physical principle is the Doppler mode: When a wave emitted at a frequency F encounters a diffuser (erythrocyte) in movement, this latter emits a wave with return frequency F±ΔF. The differential in frequency ΔF is proportional to the speed of the displacement of this diffuser. An equation then governs the relationship between this displacement speed and the Doppler frequency ΔF. Integrated in its calculator, the ultra-sonograph returns to the speed information from the Doppler frequency really measured. This speed is then represented on the Doppler image by a color whose intensity permits an evaluation of the speed from the general color scale (red and blue scale according to the direction of flow).

In order to improve the sensitivity of the detection of the vascularization (macro-circulatory), a contrast agent that enhances the backscattered signal can be injected in the organism prior to the echographic [ultrasonic] measuring.

The prior art already contains in patent WO 9219272 a contrast agent for ultrasonic exploration comprising porous particles of an inorganic material that contain a gas or a trapped liquid and whose average diameter is situated between 0.05 and 500 microns, which inorganic material is constituted by one or several substances selected from the group formed by polymeric or monomeric borates, polymeric or monomeric aluminum oxides, polymeric or monomeric carbonates, polymeric or monomeric silicas and polymeric or monomeric phosphates and pharmaceutically acceptable inorganic or organic cationic salts of these substances.

American patent NO. U.S. Pat. No. 6,203,778 also proposes a process characterizing a property of the extravascular space of a tissue using particulate contrast opaque to X-rays. This document describes a new class of particulate agents consisting of an organic or inorganic core surrounded by an organic covering of the polyethylene glycol, glucuronic acid, sialic acid type or by mixtures of these polymers.

The prior art also contains the following scientific articles:
“Magnetic Resonance Imaging Outcome after Uterine Artery Embolization for Leiomyomata with Use of Tris-Acryl Gelatin Microspheres” by F. Banovac et al,
“Micro Carrier Culture of Fibroblastic Cells on Modified Acryl Beads” by Obrenovitch et al.,
“Trisacryl Gelatin Microspheres for Therapeutic Embolization” by R. Beaujeux et al.,
“Trisacryl Gelatin Microspheres for Therapeutic Embolization” by Laurent et al.;

However, these articles disclose solutions for the embolization of tumorous vessels and the cellular culture and do not propose any interesting solution for echogenic products, that is, intended for ultrasonic exploration and/or detection.

As concerns the ultrasonic exploration and/or detection and the echogenic products intended for such uses, the products and processes of the prior art have a certain number of disadvantages. In fact, the particles are frequently unstable or have a limited lifetime and the processes for manufacturing these particles involve very significant expenses.

The ultrasonic contrast products developed and/or on the market at the present are composed by particles containing air or any gas (conferring the echogenic nature) or by perfluorinated derivatives that are matrices of hydrophobic polymers. The inventors have demonstrated for the first time the echogenic nature of a macromolecular network of hydrophilic polymers and/or of copolymers.

The inventors have now prepared microparticles that can be used as contrast agent and that do not have the disadvantages of the products of the prior art. In fact, the microparticles of the invention have the advantage of being particularly echogenic and being resistant to a prolonged ultrasonic field, that is, they have remarkable performances vis-à-vis the phenomenon of insonification.

This goal is attained by echogenic microparticles for ultrasonic exploration and/or their ultrasonic detection, characterized in that they have a size comprised between 0.1 μm (micrometer) and 2000 μm and are constituted by a macromolecular network of hydrophilic polymers and/or copolymers.

These hydrophilic polymers and copolymers advantageously comprise alcohol, amine or acid functions.

According to a first embodiment of the invention the microparticles have a size varying from 0.1 μm to 10 μm and preferably between 1 μm and 7 μm. In this instance these microparticles are used as an echographic contrast product. These microparticles can be used for the preparation of a contrast agent for ultrasonic exploration.

According to a second embodiment of the invention the microparticles have a size varying from 30 μm to 2000 μm. In this instance these microparticles are used in the viewing of the embolus by echography [ultrasonography]. These microparticles can constitute:
An implant for vascular occlusion, for filling natural cavities, for filling artificial cavities or for filling surgical cavities;
A biomaterial for tissue reconstruction.

The microparticles with a size varying from 30 μm to 2000 μm can be used as embolization particle intended for the treatment of cancers. In other words, these microparticles (from 30 μm to 2000 μm) can be used for embolus detection used for treating cancers.

The polymers and copolymers constituting the macromolecular network of the particle are preferably functionalized and/or hydrophilic derivatives such as, e.g., derivatives of alcohol, amines or acids.

These polymers and copolymers are selected from at least one of the groups comprising:
Polymeric or copolymeric acrylates and methacrylates and their derivatives, salts, esters, amides, anhydrides, nitrites, such as, e.g., diethyl amino ethane (DEAE) acrylamide, acrylamide, acrylic acid, sodium acrylate, hydroxyethyl acrylate or methacrylate;
Vinylic polymers and copolymers and their derivatives such as, e.g., vinyl acetate, vinylpyridine, vinyl sulfonates or vinyl phosphates, vinylpyrrolidone;
The polymers and copolymers of ethylene glycol and its derivatives;
The polymers and copolymers of styrene sulfonate or styrene phosphonate and their derivatives;
The polymers and copolymers of polycarboxylic acids such as the fumaric, maleic, malic, succinic, citric acids, their salts, esters, amides, anhydrides, nitrites;
The polymers and copolymers of polyethylimine and its derivatives;
The polymers and copolymers of polyvinyl sulfonate and polyvinyl phosphonate and their derivatives;
The polymers and copolymers of vinylic polyalcohol and their derivatives;
The polymers and copolymers of polyvinylpyridines, their salts and their derivatives;
The polymers and copolymers of polyvinylpyrrolidone and their derivatives.

According to a preferred embodiment of the invention the microparticles are constituted by:
N-acryloyltris (hydroxymethyl) methylamine, also called trisacryl, and by methyllene bisacrylamide (MBA),
Trisacryl, MBA and DEAE,
Sodium acrylate, MBA and DEAE,
Methacrylamide, MBA ad DEAE,
Polyvinyl alcohol (PVA).

Research work carried out within the framework of the invention permitted the documentation of the particularly interesting properties of echogenicity of the microparticles cited above.

The invention also relates to a composition for ultrasonic exploration comprising the microparticles in accordance with the invention mixed with physiological serum such as solvent in order to obtain an injectable solution.

The suspending environment used for the in-vitro experiments could consist, e.g., of a mixture of sterile water and glycerol.

Moreover, the microparticles are present in the composition at a concentration comprised between 0.25 g per liter (g/l⁻¹) and 32 g per liter (g.l⁻¹).

The invention therefore also relates to the use of the above-cited microparticles for the preparation of a composition for ultrasonic exploration in which these microparticles constitute the contrast agent.

The contrast agents in accordance with the invention and the compositions containing them are useful for the ultrasonic exploration of the human or animal body and more particularly within the framework of the study of vascularization.

Thus, the invention also concerns a process for the investigation of blood vessels and of certain organs or parts of a human or animal body such as the heart by standard echography consisting of the following stages:
The administration to the subject/patient of a dose of 0.25 to 32 grams of microparticles via the blood,
The emission of ultrasonic waves to the level of the region investigated,
The reception of the returns of ultrasonic waves stemming from the microparticles previously cited,
The calculation of the ultrasonic enhancement produced by the microparticles with the aid of an adequate software on the echo-Doppler images recorded.

In the same manner, in the case of microparticles with a size varying between 30 μm and 2000 μm the invention also relates to a process for investigating the embolization of blood vessels, characterized in that it comprises the following stages:
The administration via the blood to the subject/patient of a dose of 0.1 to 20 grams of microparticles,
The emission of ultrasonic waves to the level of the region investigated,
The reception of the returns of ultrasonic waves stemming from the microparticles previously cited,
The calculation of the ultrasonic enhancement produced by the microparticles with the aid of an adequate software for the echo-Doppler images recorded.

The invention also relates to a process for the manufacture of the microparticles previously defined and more specifically the microparticles with a size varying between 0.1 and 10 μm, characterized in that it comprises the following stages:
The realization of an inverse mini-emulsion of identical or different monomers or identical or different polymers in an organic phase comprising a surfactant by virtue of a means suitable for creating a very strong shearing or a very strong agitation or by virtue of emulsification by membrane,
The copolymerization or reticulation of the above-cited emulsion agitated in order to obtain the microparticles of polymers or copolymers such as previously described,
The recovery of the microparticles by centrifugation and re-dispersion in aqueous or organic environments.

The stage of the reticulation of the polymer chains consists in realizing these microparticles by a reagent, which polymer or polymers is/are selected from the following list:
Polymeric or copolymeric acrylates and methacrylates and their derivatives, salts, esters, amides, anhydrides, nitrites, such as, e.g., DEAE acrylamide, acrylamide, acrylic acid, sodium acrylate, hydroxyethyl acrylate or methacrylate;
Vinylic polymers and copolymers and their derivatives such as, e.g., vinyl acetate, vinylpyridine, vinyl sulfonates or vinyl phosphates, vinylpyrrolidone;
The polymers and copolymers of ethylene glycol and its derivatives;
The polymers and copolymers of styrene sulfonate or styrene phosphonate and their derivatives;
The polymers and copolymers of polycarboxylic acids such as the fumaric, maleic, malic, succinic, citric acids, their salts, esters, amides, anhydrides, nitrites;
The polymers and copolymers of polyethylene glycol or polyoxyethylene and their derivatives,
The polymers and copolymers of polyethylimine and its derivatives;
The polymers and copolymers of polystyrene sulfonate and polystyrene phosphonate and their derivatives;
The polymers and copolymers of polyvinyl sulfonate and polyvinyl phosphonate and their derivatives;
The polymers and copolymers of vinylic polyalcohol and their derivatives;
The polymers and copolymers of polyvinylpyridines, their salts and their derivatives;
The polymers and copolymers of polyvinylpyrrolidone and their derivatives.

In the same manner the reagent permitting the reticulation of the above-cited polymer(s) belongs to the following list:
Dialdehyde compounds such as, e.g., glutaraldehyde for reticulating the PVA chains and their derivatives, . . . ,
Derived dipolyacid/amine/alcohol compounds permitting a reaction of esterification with a complementary function located on the polymer chain,
Dipolyisocyanate derived compounds used, e.g., in the reticulation of chains of polyethylene glycol.

According to a possibility offered by the invention the reagent permitting the reticulation of the above-cited polymer or polymers can consist of a reagent (or coupling agent) that permits the reticulation by making two functions react that are located on two chains of different polymers, such as, e.g., the reticulation between two peptide or protein chains, e.g., the reticulation of chains of human albumin serum using EDC (1-[3-(dimethylamino)propyl]-3-ethylcarbodiimide hydrochloride).

The manufacturing process can also consist of a copolymerization of monomeric agents and reticulating monomeric agents.

In this instance the monomers can consist of one or several monomer(s) selected from the families of the following compounds:
Acrylate and methacrylate monomers, and their derivatives, salts, esters, amides, anhydrides, nitrites, . . . such as, e.g., DEAE acrylamide, acrylamide, acrylic acid, sodium acrylate, hydroxyethyl acrylate or methacrylate,

Vinylic monomers and their derivatives such as, e.g., vinyl acetate, vinyl pyridine, vinyl sulfonates or vinyl phosphates, vinylpyrrolidone,
Monomers of ethylene glycol and their derivatives,
Monomers of styrene sulfonate or styrene phosphonate and their derivatives.

As for the reticulating agent, it can consist of any substance having at least two polymerizable functions on the same molecule such as, e.g., N,N′ methylene bisacrylamide (MBA), ethylene glycol dimethacrylate (EGDM) or divinyl benzene.

On the other hand, the polymerization initiators or reticulating agent can be selected from the following list:
2,2′-azobis (2-amidino-propane)dihydrochloride, AIBN (2,2-isobutyronitrile), 2,2′-azobis(2-methyl-N-(2-hydroxyethyl)propionamide), potassium persulfate, peroxide or redox system of the type H₂O₂ and iron.

According to a variant of the process for manufacturing microparticles in accordance with the invention the process for manufacturing microparticles can consist in modifying an existing polymer chain by grafting, e.g., polymerizable functions onto it, then by causing them to react in such a manner as to obtain a coupling C—C (carbon-carbon) bond among the polymer chains. By way of example, grafting an acrylate function onto a chain of PVA and reticulating these chains by a redox system based on iron or persulfate can be envisaged.

Other advantages and characteristics of the invention will be apparent in the following examples concerning the preparation of the microparticles of the invention and their use as contrast agent in methods of ultrasonic exploration. Reference is made in these examples to the attached drawings in which:

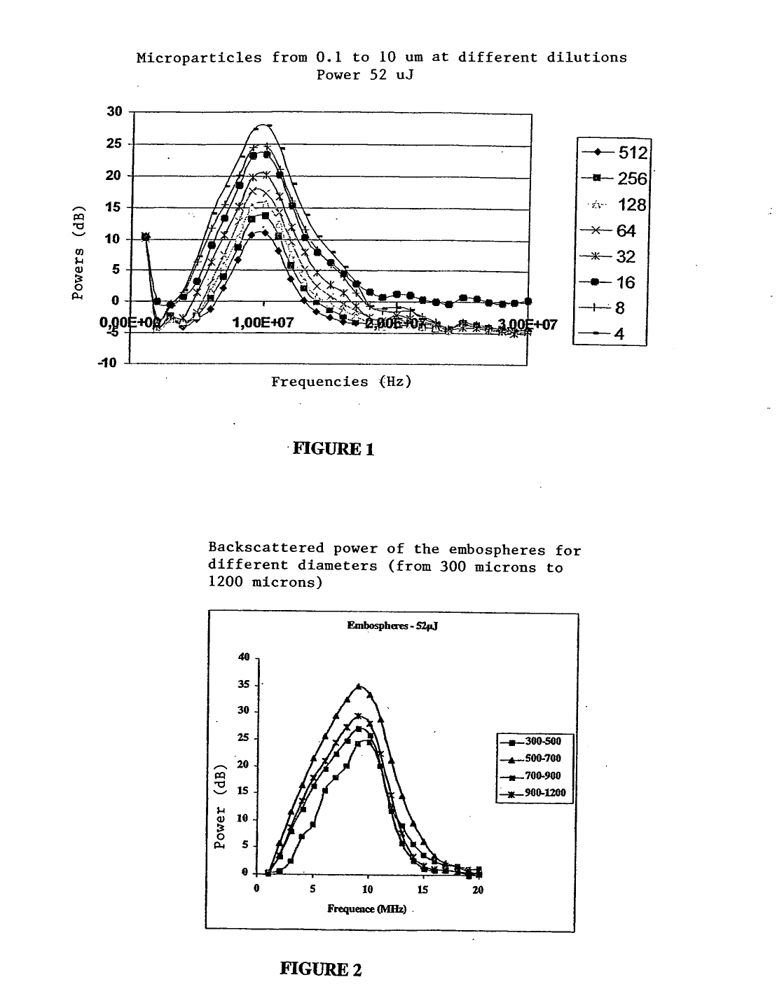

FIG. 1 is a diagram illustrating the echogenicity of the microparticles in accordance with the invention and with a size comprised between 0.1 and 10 μm for various concentrations as a function of the frequency.

FIG. 2 is a diagram illustrating the echogenicity of the microparticles in accordance with the invention and with a size comprised between 30 and 2000 μm (“embosphere” registered trademark) as a function of the frequency.

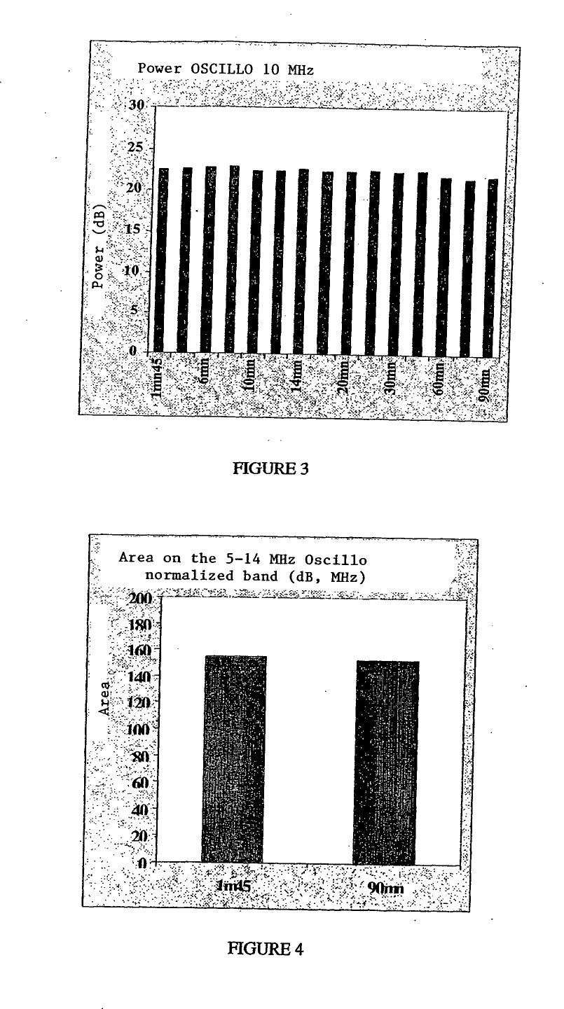

FIG. 3 is a diagram illustrating the variation of the power returned by the microparticles in accordance with the invention for an emission power at 10 MHz an a function of the time.

FIG. 4 illustrates the influence of an ultrasonic insonification at 10 kHz of repetition frequency for one and the same sample of microparticles as a function of two durations in time.

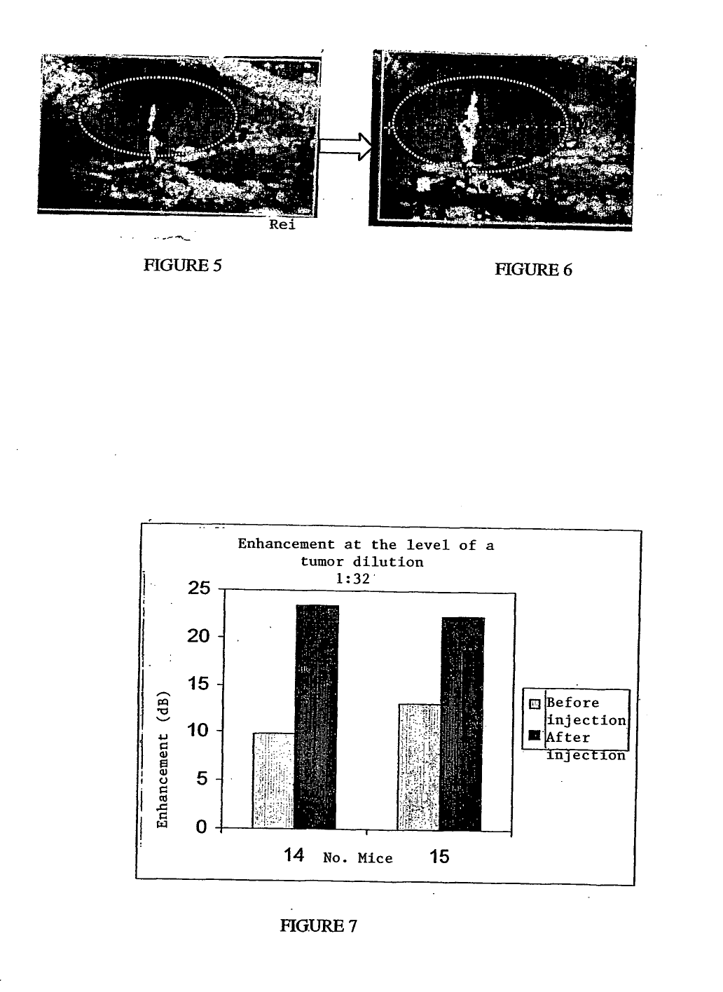

FIGS. 5 and 6 illustrate the visualization of the vascularization at the level of the kidney before (FIG. 5) and after (FIG. 6) injection in vivo of the microparticles.

FIG. 7 represents the quantification of the ultrasonic enhancement expressed in decibels (dB) produced by the microparticles at a 1/32 dilution at the level of the kidney inside the region of interest.

### EXAMPLE 1

### Manufacture of the Microparticles in Accordance with the Invention

a. Manufacture of Microparticles with a Size Comprised Between 01, and 10 μm

There are several modes of operation for realizing the spherical microparticles with the required size, a size comprised between 0.1 and 10 micrometers (μm). Thus, the size of the microparticles is a function of several parameters of which the principal ones are the agitation speed, the concentration of surfactant and the ratio of viscosity between the two environments that are the monomer phase (aqueous phase) and the continuous phase (organic phase).

An example of manufacture of microparticles in accordance with the invention by a polymerization in mini-emulsion will now be described in the following that is characterized by very elevated agitation speeds and the concentration of surfactant greater than the polymerization in suspension in order to form a pre-emulsion. Thus, the polymerization is then carried out on this pre-emulsion.

One liter of heptane and 4 milliliters of sorbitan trioleate are introduced into a two-liter reactor. This solution is brought to a temperature of 40° C. (Celsius) under very vigorous agitation and an aqueous solution of 125 g (g) of trisacryl, 29 g methylene bis-acrylamide and 2.51 g 2,2′-azobis(2-amidino-propane) dihydrochloride (or V-50) is introduced. After fifteen minutes the agitation is stopped and this emulsion is brought to a temperature of 80° C. for four hours. The resulting dispersion is washed with hexane and dried. The microparticles are then redispersed in a saline solution.

The same mode of operation was used for synthesizing microparticles based on methacrylamide and DEAE acrylamide.

b. Manufacture of Microparticles with a Size Comprised Between 30 and 2000 μm

In order to manufacture microparticles with a size comprised between 30 micrometers (μm) and 2000 μm for their use in the visualization of a vascular embolus by echography two modes of operation are presented below: One for realizing microparticles based on trisacryl with porcine gelatin and the other for realizing microparticles based on sodium acrylate.

For the microparticles based on trisacryl with porcine gelatin (“Embosphere” registered trademark), a 10-liter beaker is used. 4 liters of paraffin oil and 4 ml of sorbitan trioleate are poured in and the beaker heated on a water bath between 54° C. and 60° C. (Celsius).

87 g of sodium chloride and 40.8 g of sodium acetate are weighed in a 1-liter beaker. They are put in solution in 300 ml of demineralized water. Then, 400 ml glycerol and the different monomers trisacryl (90 grams), DEAE acrylamide (35 grams) and methylene bis acrylamide (10 grams) are added.

On the other hand, the gelatin is put in suspension in 120 ml ultra-pure water and heated to 60° C.

When the monomers are dissolved, the solution of gelatin and 1.4 g ammonium persulfate, previously dissolved in 20 ml of ultra-pure water are added. The monomeric phase is then poured into the oily phase at 60° C. under agitation. Then, 4 ml N,N,N′,N′-tetraethylmethanediamine (TEMED) are poured into the emulsion.

The microparticles are then recovered by decantation and carefully washed. These microparticles are then treated with glutaraldehyde and washed several times at 60 to 90° C.

They are then sieved and sterilized with vapor in a buffered environment.

A 10-liter beaker is also user for microparticles based on sodium acrylate.

The operator pours 4 liters of paraffin oil, 4 ml of sorbitan trioleate and heats the mixture on a water bath at a temperature comprised between 54° C. and 60°.

87 g of sodium chloride and 40.8 g of sodium acetate are weighed in a 1-liter beaker. They are put in solution in 300 ml of demineralized water. Then, 400 ml glycerol are added. Sodium acrylate (104 g) and methylene bis acrylamide (27.5 grams) are added.

When the monomers are dissolved, the solution of gelatin and 1.4 g ammonium persulfate, previously dissolved in 20 ml of ultra-pure water are added. The monomeric phase is then poured into the oily phase at 60° C. under agitation. Then, 4 ml TEMED is poured into the emulsion.

The microparticles are then recovered by decantation and carefully washed. They are then sieved and sterilized with vapor in a buffered environment.

### EXAMPLE 2

### Result and In Vitro Studies Relative to the Echogenic Properties of the Microparticles in Accordance with the Invention

a) Echogenicity of Microparticles with a Size Comprised Between 0.1 and 10 μm

As can be seen in FIG. 1, 8 dilutions of microparticles were studied, namely, 1/512, 1/256, 1/128, 1/64, 1/32, 1/16, 1/8 and 1/4. These dilutions were calculated relative to an initial concentration fixed at 5 g of microparticles in accordance with the invention in 40 ml of suspending environment.

The graph of FIG. 1 represents the different curves for each of the above-cited concentrations as a function of the frequency. Thus, as can be seen in this figure:
The backscattered power increases with the concentration and
The power backscattered by the particles is greater than 10 dB for the 1/512 dilution and increases until attaining 25 dB for the greatest concentration at 52 μJ.

The particles whose size is comprised between 0.1 and 10 μm therefore have strong properties of echogenicity.

b. Echogenicity of the Microparticles with a Size Comprised Between 30 and 2000 μm (“Embosphere” Registered Trademark)

FIG. 2 shows the spectra representing the backscattered power of the embosphere particles with a power of 52 μJ. 4 ranges of sizes were studied: from 300 to 500 μm, from 500 to 700 μm, from 700 to 900 μm and from 900 to 1200 μm.

Influence of Ultrasound Insonification on the Integrity of Microparticles:

The properties of the “PRII10” microparticles (compounds of trisacryl and MBA) were then tested vis-à-vis the phenomenon of insonification. The ⅛^{th dilution was used in order to do this.}

During this protocol the microparticles present in the suspension were insonified with a wave of 10 MHz with a repetition frequency of 1 kHz. The backscatter measurements were taken at different time intervals. The results of these measurements are shown in FIG. 3.

It was observed that the ultrasonic backscatter does not vary in a significant manner in time when the microparticles of the invention are subjected to an ultrasonic field in a prolonged manner. As a consequence, it appears that the microparticles were not altered or destroyed under the effect of an insonification of given and prolonged energy.

Taking the previous results into account, the decision was made to pursue the study for an ultrasonic energy emitted at a higher level. Since the amplitude of the emitted wave was regulated on the emitter at its maximum level, the repetition frequency of the wave at 10 kHz was increased. In this manner the ultrasonic power transmitted to the particles is globally more significant for an equivalent time interval.

As FIG. 4 shows, it appears that the maximum available level of power emitted is insufficient for destroying the microparticles even partially. In fact, the power obtained 90 minutes after the start of the protocol is identical to that taken only 1.45 minutes after the start of the insonification.

Finally, the choice was made to test the influence of the shearing rate on the resistance of the microparticles. Thus, in this test the wave was emitted only at the moment of performing the measurement and two measurements were taken, at 1.45 minutes and 90 minutes after the triggering of the circulation of the suspension.

As for the influence of a prolonged ultrasonic insonification, we did not observe any significant variation of the ultrasonic backscattering of the microparticles when they are submitted to a constant and durable shearing field. Thus, it can be concluded that the microparticles are not only resistant to the ultrasonic wave employed but also to a mechanical shearing field.

Stability tests were performed in vitro on the microparticles of the invention. These microparticles were submitted to a shearing field and to an ultrasonic field, both applied in a prolonged manner, and the echogenicity of the microparticles remained constant during the entire duration of the experiment, that is, for two hours. Inversely, Levovist is much less stable: The enhancement peak is produced quasi instantaneously after the injection of the product and then diminishes progressively until the complete disappearance 10 min after the injection. This latter point is a limiting factor during an echographic examination.

These tests and studies permitted the echogenic nature of the microparticles of the invention to be documented. Furthermore, these microparticles seem simultaneously resistant to an ultrasonic field and to a shearing field both applied in a prolonged manner.

### EXAMPLE 3

### Quantification of the Ultrasonic Enhancement of the Particles whose Size is Comprised Between 0.1 and 10 μm in Different Dilutions In Vivo in Nude Mice

Course of In-In Vivo Injections:

Suspension Environment used In Vivo
The suspending environment used was composed of 100% sterile water+9°/∞NaCl

Device Used
The explorations on animals were realized with an ATL HDI5000 ultrasonograph. This device is connected to a research software (HDILab) for the post-quantification of the ultrasonic enhancement in a selected region of interest.

Conditions
The mice used were nude (hairless immuno-depressive), facilitating the echographic trials in a first period. The majority of the mice do not have a tumor except when stated (see table) and the enhancement was observed principally at the level of the kidneys and the spleen.

The injections were performed with the following dilutions:
Dilution 1/64: 1.95 mg CCII03-14/ml
Dilution 1/32: 3.9 mg CCII03-14 ml
Dilution 1/16: 7.8125 mg CCII03-14 ml

Each injection corresponded to 0.1 ml, that is, 0.195 mg, 0.39 mg and 0.78125 mg respectively for the dilutions 1/64, 1/32 and 1/16.

The syringes systematically had a diameter of 30 μm.

The following table recapitulates the conditions of injections and of sacrifices:

TABLE 1

| No. | | | Day | Day | Waiting |
|---|---|---|---|---|---|
| group | No. mice | Dilution | injection | sacrifice | period |
| 1 | 1 | 64 | April 25 | April 30 | 1 week |
| 1 | 2 | 64 | April 25 | April 30 | 1 weeks |
| 2 | 1 | 64 | April 29 | May 5 | 1 week |
| 2 | 2 | 64 | April 29 | May 5 | 1 week |
| 2 | 3 | 64 | April 30 | May 15 | 2 weeks |
| 2 | 4 | 64 | April 30 | May 15 | s |
| 3 | 1 (male) | 64 | May 16 | June 6 | 3 weeks |
| 3 | 2 (male) | 32 | May 16 | May 23 | 1 week |
| 3 | 3 (male) | 32 | May 16 | May 23 | 1 week |
| 4 | 1 | 64 | May 20 | June 10 | 3 weeks |
| 4 | 2 | 32 | May 20 | June 5 | 2 weeks |
| 4 | 3 | 32 | May 20 | June 5 | 2 weeks |
| 4 | 4 (tumor) | 32 | May 20 | June 11 | 3 weeks |
| 5 | 1 (tumor) | 32 | May 28 | June 17 | 3 weeks |
| 5 | 2 (tumor) | 16 | May 28 | June 5 | 1 weeks |

TABLE 2
The table represents the detection of vessels before and after injection
in vivo of the microparticles in nude mice. The vessels were counted
by analysis of the echo-Doppler images recorded.

| Dilution 1/64 | | | | Dilution 1/32 | | | |
|---|---|---|---|---|---|---|---|
| No. | Before | After | | No. | Before | After | |
| mice | injection | injection | Difference | mice | injection | injection | Difference |
| 1 | 6 | 7 | 1 | 8 | 5 | 5 | 0 |
| 2 | 6 | 8 | 2 | 9 | 5 | 7 | 2 |
| 3 | 8 | 12 | 4 | 11 | 6 | 8 | 2 |
| 4 | 7 | 9 | 2 | 12 | 7 | 7 | 0 |
| 5 | 8 | 12 | 4 | 13 | 7 | 7 | 0 |
| 6 | 7 | 9 | 2 | 13 | Transversal | Transversal | 2 |
| | | | | | tumor: 2 | tumor: 4 | |
| 7 | 8 | 8 | 0 | 14 | 6 | 8 | 2 |
| 10 | 7 | 8 | 1 | 14 | Tumor TR: 4 | Tumor TR: 11 | 7 |

Results

The results were expressed in two ways:

1—By calculating the ultrasonic enhancement produced by the microparticles inside a region of interest:
The kidney was chosen as region of interest (ROI) (represented in dotted lines in FIGS. 5, 6) for each mouse. The contours of the ROI were designed on each of the echographic sequences with the aid of the HDILab software. The echogenicity was therefore quantified before and after injection inside the ROI. FIG. 7 shows an example of enhancements obtained at the level of a xenografted tumor on two mice at dilution 1/32.

2—By counting the vessels inside the kidney:
A better detection of the vascularization after injection of the microparticles was determined in almost all instances. FIGS. 5, 6 show an example of echo-Doppler images recorded before and after injection of the microparticles. The detection of the vessels is distinctly improved after injection. It was possible from these images to count the vessels detected before and after injection. The second table summarizes the results obtained for two study dilutions: 1/64 and 1/32. The average number of supplementary vessels detected after injection of the particles is on the average 2 or 3 vessels.

### EXAMPLE 4

### The Use or Implementation of the Microparticles

When a patient requires a classic echographic-Doppler examination the procedure is as follows:
The probe is placed in contact with the patient's skin at the location where the examination is to be performed. An echographic gel is spread on the surface of the probe beforehand in order to assure a good transmission of the ultrasonic beam between the probe and the tissues,
The measuring depth as well as the bidimensional gain of the ultrasonograph are respectively regulated in accordance with the depth at which the organ of interest is located and the contrast of the image obtained,
When the organ of interest has been located on the image the examiner proceeds to measure the dimensions of the organ on the arrested image. A photograph is generally printed and the image is digitally stored,
If a visualization of the vessels is necessary, the color Doppler mode is activated. A sector then appears on the image that is correctly positioned by the operator in order to superpose it at the level of the vessels of interest,
The maximum measurable speed is regulated by the operator in such a manner as not to generate measuring errors (aliasing),
The operator engages the pulsed Doppler in order to obtain the triplex mode. This permits a precise measurement volume to be defined in the flow and the speed spectrum to be obtained in the course of time for measuring the circulatory speeds.

By way of example, echography permits a descriptive analysis of the morphology and of the structure of the thyroid. It permits the evaluation of:
The dimensions of each lobe (height, thickness and width),
Its contours,
Any nodules and the study of their characteristics (echostructure, echogenicity),
The ganglionic areas: Size, aspect and situation of any adenopathies,
Any compressions and deformations of the adjacent organs,

And it might permit:
The study of the thyroid vascularization,
The guiding of the cytopuncture of a palpable or poorly palpable thyroid nodule.

For this type of investigation a high-frequency probe (7.5 MHz or more) is indispensable for obtaining a high spatial resolution (linear array [strip]). The device should be regularly updated technically and have a quality control. A large-size linear probe or a sector probe permitting the study of diving goiters and the measuring of the height of the lobes of a goiter.

The microparticles are preferably administered to the patient via the blood directly where the echography is to be performed. The dose administered is variable as a function of the patient. For a small animals such as a mouse, e.g., 0.2 milliliters of an emulsion are administered with a concentration of 0.8 grams of microparticles in accordance with the invention in 40 milliliters of suspending environment, that is, a concentration of 2 grams of microparticles per liter solvent. The solvent is constituted, e.g., by physiological serum that of course does not contain any backscattering element.

1-23. (canceled)

24. A composition for ultrasonic exploration comprising echogenic microparticles comprising a macromolecular network of reticulated hydrophilic polymers and/or copolymers which microparticles have a diameter between 0.1 μm and 10 μm and are obtained from an inverse mini-emulsion of identical or different monomers or polymers.

25. The composition according to claim 24, wherein the microparticles have a diameter between 1 μm and 7 μm.

26. The composition according to claim 24, wherein the hydrophilic polymers and copolymers are alcohol, amine or acid.

27. The composition according to claim 24, wherein the polymers and copolymers are selected from at least the groups consisting of:
polymeric or copolymeric acrylates and methacrylates, their derivatives, salts, esters, amides, anhydrides and nitrites;
vinylic polymers, copolymers and their derivatives;
polymers and copolymers of ethylene glycol and their derivatives;
polymers and copolymers of styrene syulfonate, styrene phosphonate and their derivatives;
polymers and copolymers of polycarboxylic, acids, their salts, esters, amides, anhydrides and nitrites;
polymers and copolymers of polyethylimine and their derivatives;
polymers and copolymers of polyvinyl sulfonate and polyvinyl phosphonate and their derivatives;
polymers and copolymers of vinylic polyalcohol and their derivatives;
polymers and copolymers of polyvinylpyridines, their salts and their derivatives; and
polymers and copolymers of polyvinylpyrrolidone and their derivatives.

28. The composition according to claim 24, wherein the microparticles comprise N-acryloyltris (hydroxymethyl)methylamine, and methylene bisacrylamide (MBA).

29. The composition according to claim 24, further comprising a suspending environment.

30. The composition according to claim 29, wherein the suspending environment comprises a mixture of sterile water and glycerol or physiological serum.

31. The composition according to claim 30, wherein the microparticles are present at a concentration between 0.25 g per liter (g/l⁻¹) and 32 g per liter (g.l⁻¹).

32. A process for investigating blood vessels and organs or parts of a human or animal body by standard echography and/or Doppler echography comprising:
systemic administration to a subject/patient of a dose with a composition according to claim 24,
emitting ultrasonic waves toward a target region with an ultrasonic wave generator,
receiving returning ultrasonic waves stemming from microparticles, and
calculating ultrasonic enhancement produced by the microparticles on echo-Doppler images recorded.

33. A process for investigating embolization of blood vessels comprising:
systemic administration to a subject/patient of a dose of a composition according to claim 24,
emitting ultrasonic waves toward a target region with an ultrasonic wave generator,
receiving returning ultrasonic waves stemming from the microparticles, and
calculating ultrasonic enhancement produced by the microparticles on echo-Doppler images recorded.

34. A contrast agent for ultrasonic exploration comprising echogenic microparticles comprising a macromolecular network of reticulated, hydrophilic polymers and/or copolymers, which microparticles have a diameter between 0.1 μm and 10 μm.

35. A process for manufacturing microparticles defined in claim 24, comprising:
preparing an inverse mini-emulsion of identical or different monomers or identical or different polymers in an organic phase comprising a surfactant with strong shearing, strong agitation or emulsification by membrane,
copolymerization or reticulation of the emulsion to obtain the microparticles of polymers or copolymers, and
recovering the microparticles by centrifugation and re-dispersion in aqueous or organic environments.

36. The process according to claim 35, wherein polymerization is realized by a reagent, which polymer or polymers is/are selected from the groups consisting of:
polymeric or copolymeric acrylates and methacrylates and their derivatives, salts, esters, amides, anhydrides and nitrites;
vinylic polymers and copolymers and their derivatives;
polymers and copolymers of ethylene glycol and their derivatives;
polymers and copolymers of styrene sulfonate, styrene phosphonate and their derivatives;
polymers and copolymers of polycarboxylic acids, their salts, esters, amides, anhydrides and nitrites;
polymers and copolymers of polyethylene glycol, polyoxyethylene and their derivatives;
polymers and copolymers of polyethylimine and their derivatives;
polymers and copolymers of polystyrene sulfonate, polystyrene phosphonate and their derivatives;
polymers and copolymers of polyvinyl sulfonate, polyvinyl phosphonate and their derivatives;
polymers and copolymers of vinylic polyalcohol and their derivatives;
polymers and copolymers of polyvinylpyridines, their salts and their derivatives; and
polymers and copolymers of polyvinylpyrrolidone and their derivatives.

37. The process according to claim 36, wherein the reagent permitting reticulation of the polymer(s) is selected from the group consisting of:
dialdehyde compounds and their derivatives,
derived dipolyacid/amine/alcohol compounds permitting a reaction of esterification with a complementary function located on the polymer chain, and
dipolyisocyanate derived compounds.

38. The process according to claim 37, wherein the reagent comprises a reactant permitting reticulation by making two functions react that are located on two chains of different polymers using EDC (1-[3-(dimethylamino)propyl]-3-ethylcarbodiimide hydrochloride).

39. The process defined in claim 35, further comprising copolymerization of monomeric agents and reticulating agents.

40. The process according to claim 39, wherein the monomers consist of one or several monomer(s) selected from the groups consisting of:
acrylate and methacrylate monomers, their derivatives, salts, esters, amides, anhydrides and nitrites,
vinylic monomers and their derivatives,
monomers of ethylene glycol and their derivatives, and
monomers of styrene sulfonate, styrene phosphonate and their derivatives.

41. The process according to claim 39, wherein the reticulating agent comprises any substance having at least two polymerizable functions on the same molecule.

42. The process according to claim 39, wherein the reticulating agent or polymerization initiator comprises an agent sleected from the group consisting of: 2,2′-azobis (2-amidino-propane)dihydrochloride, AIBN (2,2′-azobisisobutyronitrile), 2,2′-azobis(2-methyl-N-(2-hydroxyethyl)propionamide), potassium persulfate, peroxide or redox system of the type H₂O₂ and iron.

43. Echogenic microparticles comprising a macromolecular network of reticulated hydrophilic polymers and/or copolymers, which microparticles have a diameter between 0.1 μm and 10 μm, and are obtained from an inverse mini-emulsion of identical or different monomers or polymers.

44. The microparticles according to claim 43, wherein the hydrophilic polymers and copolymers are alcohol, amine or acid functions.

45. The microparticles according to claim 43, wherein the polymers and copolymers are selected from at least the groups consisting of:
polymeric or copolymeric acrylates and methacrylates, their derivatives, salts, esters, amides, anhydrides and nitrites;
vinylic polymers, copolymers and their derivatives;
polymers and copolymers of ethylene glycol and their derivatives;
polymers and copolymers of styrene sulfonate, styrene phosphonate and their derivatives;
polymers and copolymers of polycarboxylic acids, their salts, esters, amides, anhydrides and nitrites;
polymers and copolymers of polyethylimine and their derivatives;
polymers and copolymers of polyvinyl sulfonate, polyvinyl phosphonate and their derivatives;
polymers and copolymers of vinylic polyalcohol and their derivatives;
polymers and copolymers of polyvinylpyridines, their salts and their derivatives; and
polymers and copolymers of polyvinylpyrrolidone and their derivatives.

46. The microparticles according to claim 43, comprising N-acryloyltris (hydroxymethyl) methylamine, and methylene bisacrylamide (MBA).
